(19) United States
(12) Patent Application Publication
(10) Pub. No.: US 2016/0183985 A1
(43) Pub. Date: Jun. 30, 2016

(54) SPINAL STABILIZATION CAM-LOCKING CROSS-CONNECTOR

(71) Applicant: SPINAL USA, INC., Parsippany, NJ (US)

(72) Inventor: Michael N. Campbell, Elmwood Park, NJ (US)

(21) Appl. No.: 14/909,039

(22) PCT Filed: Jul. 24, 2014
(86) PCT No.: PCT/US2014/048037

§ 371 Date: Jan. 29, 2016

Related U.S. Application Data
(60) Provisional application No. 61/860,574, filed on Jul. 31, 2013.

(51) Int. Cl. A61B 17/70

(52) U.S. Cl. CPC A61B 17/7049; A61B 2560/0443; A61B 2560/045

(57) ABSTRACT

An adjustable spinal implant device including a first member and a second member, the first member adapted to receive the second member and to lock the second member in position relative to the first member. The first and second members may each comprise coupling portions adapted to couple to a spinal stabilization rod with a snap-fit. Cam locks may be adapted to be rotatably positioned in each member, wherein each member comprises a cam bore configured to receive a cam lock. The first cam lock and second cam lock may be each rotatable between a locked position and an open position.

### CROSS-REFERENCE TO RELATED APPLICATIONS

This application claims priority benefit of U.S. Provisional Application No. 61/860,574, entitled “SPINAL STABILIZATION CAM-LOCKING CROSS-CONNECTOR,” filed Jul. 31, 2013, the entirety of which is hereby incorporated by reference.

### TECHNICAL FIELD

The present application relates to systems, devices, and methods for spinal surgeries. In particular, the present application relates to systems, devices, and methods for spinal stabilization.

### DESCRIPTION OF THE RELATED TECHNOLOGY

Referring to FIG. 1 a perspective view of the spinal anatomy is shown. The spinal column 100 is divided into five sections beginning with the cervical section 102, the thoracic section 104, the lumbar section 106, the sacrum 108 and finally the coccyx 110. Each major section (cervical 102, thoracic 104, lumbar 106) is made up of individual bones called vertebrae. In a conventional spinal configuration, there are 7 cervical vertebrae, 12 thoracic vertebrae, and 5 lumbar vertebrae.

An individual vertebra is made up of several anatomical features. Generally, each vertebra of the three major sections has the same major features. FIG. 2 is a cross-sectional view of a vertebra 200. The body 202 of the vertebra is the primary area of weight bearing and provides a resting place for the fibrous discs which separate each of the vertebrae. The lamina 208 covers the spinal canal, the large opening in the center of the vertebra through which the spinal cord passes, and the neural foramen, where the spinal nerves exit. The spinous process 212 is the bone that can be felt when running a hand down a person's back. The paired transverse processes 204 are oriented 90 degrees to the spinous process 212 and provide attachment for back muscles. Pedicles 206 connect the transverse processes 204 to the body 202. Located between the transverse processes 204 and the laminae 208 are superior articular processes 210.

In some situations, pedicle screws and stabilization rods can be utilized to limit the movement between vertebrae 200 and stabilize the spine 100. The pedicle screws can be driven into the pedicles 206 of each vertebrae 200 and are configured to accept a spinal stabilization rod. A set screw can be utilized to retain the stabilization rod within a portion of the pedicle screw and limit movement of the vertebra in relation to the stabilization rod. The stabilization rod can be affixed to additional pedicle screws installed in other vertebrae, effectively limiting movement between the vertebrae 200. Sometimes, multiple stabilization rods are utilized and it may be preferable to limit movement between each stabilization rod.

### SUMMARY

The systems, methods and devices described herein have innovative aspects, no single one of which is indispensable or solely responsible for their desirable attributes. Without limiting the scope of the claims, some of the advantageous features will now be summarized.

One aspect of the present invention is the realization that existing systems, devices, and methods for limiting movement between stabilization rods fail to easily and effectively couple to each stabilization rod and limit movement between them. Thus, there exists a need for a spinal stabilization cam-locking cross-connector which easily and effectively couples to each stabilization rod and limits movement between them.

One non-limiting embodiment of the present invention includes a connector configured to link to lateral stabilization rods in pedicle screw based posterior fusion of the thoracolumbar spinal column and facilitate rigid fixation between the lateral stabilization rods.

In another embodiment, an adjustable spinal implant device can include a first member and a second member, the first member adapted to receive the second member and to lock the second member in position relative to the first member, wherein the first member comprises a first coupling portion adapted to couple to a first spinal stabilization rod with a snap-fit, wherein the second member comprises a second coupling portion adapted to couple to a second spinal stabilization rod with a snap-fit, wherein the first coupling portion comprises a first rod recess adapted to receive the first spinal stabilization rod, wherein the second coupling portion comprises a second rod recess adapted to receive the second spinal stabilization rod, a first cam lock adapted to be rotatably positioned in the first member, a second cam lock, adapted to be rotatably positioned in the second member, wherein the first member comprises a first cam bore configured to receive a first cam lock, wherein the second member comprises a second cam bore configured to receive a second cam lock, wherein the first cam lock and second cam lock are each rotatable between a locked position and an open position, wherein the first rod recess of the first coupling portion is adapted to receive the first spinal stabilization rod when the first cam lock is in an open position, wherein the first member is adapted to block the first spinal stabilization rod from exiting the rod recess of the first coupling portion when the first cam lock is in a locked position, wherein the second rod recess of the second coupling portion is adapted to receive the second spinal stabilization rod when the second cam lock is in an open position, and wherein the second member is adapted to block the second spinal stabilization rod from exiting the rod recess of the second coupling portion when the second cam lock is in a locked position.

In another embodiment, the first member comprises a slide lock adapted to receive the second member, wherein the second member is slidably and rotatably positionable within the slide lock.

In another embodiment, the first coupling portion comprises a first hook portion, the first hook portion adapted to surround at least a portion of the first rod recess, and wherein the second coupling portion comprises a second hook portion, the second hook portion adapted to surround at least a portion of the second rod recess.

In another embodiment, the first hook portion is adapted to deflect from a default orientation when the first spinal stabilization rod enters the first rod recess and to return towards the default orientation when the first stabilization rod is fully seated in the first rod recess, and wherein the second hook portion is adapted to deflect from a default orientation when the second spinal stabilization rod enters the second rod recess and to return towards the default orientation when the second stabilization rod is fully seated in the second rod recess.

In another embodiment, the first hook portion is adapted to retain the first spinal stabilization rod in the first rod recess when the first cam lock is in an open position and where the second hook portion is adapted to retain the second spinal stabilization rod in the second rod recess when the second cam lock is in an open position.

In another embodiment, the first cam lock comprises a first protrusion adapted to engage the first spinal stabilization rod when the first cam lock is in a locked position, the first protrusion adapted to block the first spinal stabilization rod from exiting the first rod recess, and wherein the second cam lock comprises a second protrusion adapted to engage the second spinal stabilization rod when the second cam lock is in a locked position, the second protrusion adapted to block the second spinal stabilization rod from exiting the second rod recess.

In another embodiment, the first protrusion and the second protrusion each comprise a rod engagement surface.

In another embodiment, rotation of the first cam lock and the second cam lock from an open position to a locked position requires no more than approximately 90 degrees of rotation.

In another embodiment, rotation of the first cam lock and the second cam lock from an open position to a locked position requires no more than approximately 100 degrees of rotation.

In another embodiment, rotation of the first cam lock and the second cam lock from an open position to a locked position requires between approximately 80 and 100 degrees of rotation.

In another embodiment, rotation of the first cam lock and the second cam lock from an open position to a locked position requires between approximately 70 and 110 degrees of rotation.

In another embodiment, rotation of the first cam lock and the second cam lock from an open position to a locked position requires between approximately 60 and 120 degrees of rotation.

In another embodiment, rotation of the first cam lock and the second cam lock from an open position to a locked position requires between approximately 50 and 130 degrees of rotation.

In another embodiment, an adjustable spinal implant device can include a first member and a second member, the first member comprising a slide lock adapted to receive the second member, wherein the second member is slidably and rotatably positionable within the slide lock, wherein the first member comprises a first coupling portion adapted to couple to a first spinal stabilization rod, wherein the second member comprises a second coupling portion adapted to couple to a second spinal stabilization rod, wherein the first coupling portion comprises a first rod recess adapted to receive the first spinal stabilization rod, wherein the second coupling portion comprises a second rod recess adapted to receive the second spinal stabilization rod, a first cam lock adapted to be rotatably positioned in the first member, a second cam lock, adapted to be rotatably positioned in the second member, wherein the first member comprises a first cam bore configured to receive a first cam lock, wherein the second member comprises a second cam bore configured to receive a second cam lock, wherein the first cam lock and second cam lock are each rotatable between a locked position and an open position, wherein the first rod recess of the first coupling portion is adapted to receive the first spinal stabilization rod when the first cam lock is in an open position, wherein the first member is adapted to block the first spinal stabilization rod from exiting the rod recess of the first coupling portion when the first cam lock is in a locked position, wherein the second rod recess of the second coupling portion is adapted to receive the second spinal stabilization rod when the second cam lock is in an open position, and wherein the second member is adapted to block the second spinal stabilization rod from exiting the rod recess of the second coupling portion when the second cam lock is in a locked position, wherein rotation of the first cam lock and the second cam lock from a open position to a locked position requires no more than approximately 90 degrees of rotation.

In another embodiment, rotation of the first cam lock and the second cam lock from an open position to a locked position requires no more than approximately 100 degrees of rotation.

In another embodiment, rotation of the first cam lock and the second cam lock from an open position to a locked position requires between approximately 80 and 100 degrees of rotation.

In another embodiment, rotation of the first cam lock and the second cam lock from an open position to a locked position requires between approximately 70 and 110 degrees of rotation.

In another embodiment, rotation of the first cam lock and the second cam lock from an open position to a locked position requires between approximately 60 and 120 degrees of rotation.

In another embodiment, rotation of the first cam lock and the second cam lock from an open position to a locked position requires between approximately 50 and 130 degrees of rotation.

In another embodiment, the first cam lock comprises a first protrusion adapted to engage the first spinal stabilization rod when the first cam lock is in a locked position, the first protrusion adapted to block the first spinal stabilization rod from exiting the first rod recess, and wherein the second cam lock comprises a second protrusion adapted to engage the second spinal stabilization rod when the second cam lock is in a locked position, the second protrusion adapted to block the second spinal stabilization rod from exiting the second rod recess.

In another embodiment, the first protrusion and the second protrusion each comprise a curved rod engagement surface

In another embodiment, wherein the first member comprises a first coupling portion adapted to couple to a first spinal stabilization rod with a snap-fit, and wherein the second member comprises a second coupling portion adapted to couple to a second spinal stabilization rod with a snap-fit.

In another embodiment, wherein the first coupling portion comprises a first hook portion, the first hook portion adapted to surround at least a portion of the first rod recess, and wherein the second coupling portion comprises a second hook portion, the second hook portion adapted to surround at least a portion of the second rod recess.

In another embodiment, wherein the first hook portion is adapted to deflect from a default orientation when the first spinal stabilization rod enters the first rod recess and to return towards the default orientation when the first stabilization rod is fully seated in the first rod recess, and wherein the second hook portion is adapted to deflect from a default orientation when the second spinal stabilization rod enters the second rod recess and to return towards the default orientation when the second stabilization rod is fully seated in the second rod recess.

In another embodiment, wherein the first hook portion is adapted to retain the first spinal stabilization rod in the first rod recess when the first cam lock is in an open position and where the second hook portion is adapted to retain the second spinal stabilization rod in the second rod recess when the second cam lock is in an open position.

In another embodiment, a method of stabilizing the spine can include coupling a first member of a connector to a first spinal stabilization rod, coupling a second member of the connector to a second spinal stabilization rod, rotating a first cam lock approximately 90 degrees from an open position to a locked position, and rotating a second cam lock approximately 90 degrees from an open position to a locked position.

In another embodiment, the method can include comprising locking the first member to the second member, limiting movement and rotation between the first member and the second member.

In another embodiment, coupling the first member of the connector to the first spinal stabilization rod comprises forcing the first spinal stabilization rod into a first rod recess of the first member until a portion of the first member deflects and the first spinal stabilization rod enters a first rod recess of the first member, and wherein coupling the second member of the connector to the second spinal stabilization rod comprises forcing the second spinal stabilization rod into a second rod recess of the second member until a portion of the second member deflects and the second spinal stabilization rod enters the second rod recess of the second member.

In another embodiment, coupling the first member to the first spinal stabilization rod produces a snap-fit, and wherein coupling the second member to the second spinal stabilization rod produces a snap-fit.

In another embodiment, coupling the first member to the first spinal stabilization rod produces an abrupt movement of the first member relative to the first spinal stabilization rod, and wherein coupling the second member to the second spinal stabilization rod produces an abrupt movement of the second member relative to the second spinal stabilization rod.

In another embodiment, coupling the first member to the first spinal stabilization rod produces a click, and wherein coupling the second member to the second spinal stabilization rod produces a click.

### BRIEF DESCRIPTION OF THE DRAWINGS

The above-mentioned aspects, as well as other features, aspects, and advantages of the present technology will now be described in connection with various embodiments, with reference to the accompanying drawings. The illustrated embodiments, however, are merely examples and are not intended to be limiting. Like reference numbers and designations in the various drawings indicate like elements.

FIG. 1 illustrates a side view of the spinal anatomy.

FIG. 2 illustrates a top view of a vertebra.

FIG. 3 illustrates a perspective view of one embodiment of a connector coupled to lateral stabilization rods of a spinal stabilization system.

FIG. 4 illustrates an exploded perspective view of one embodiment of a connector.

FIG. 5 illustrates a perspective view of one embodiment of a first member.

FIG. 6 illustrates a perspective view of one embodiment of a second member.

FIG. 7 illustrates an exploded perspective view of one embodiment of a slide lock.

FIG. 8A illustrates a side view of a connector in a short configuration.

FIG. 8B illustrates a side view of a connector in a long configuration.

FIG. 9A-B illustrate partial perspective views of one embodiment of a coupling portion.

FIG. 10 illustrates a cross-sectional view of one embodiment of a coupling portion and a cam lock in a locked position.

FIG. 11A-B illustrate perspective views of one embodiment of a cam lock.

FIG. 11C illustrates a side view of the cam lock of FIGS. 11A-B.

FIG. 11D illustrates a cross-sectional view of the cam lock of FIGS. 11A-B.

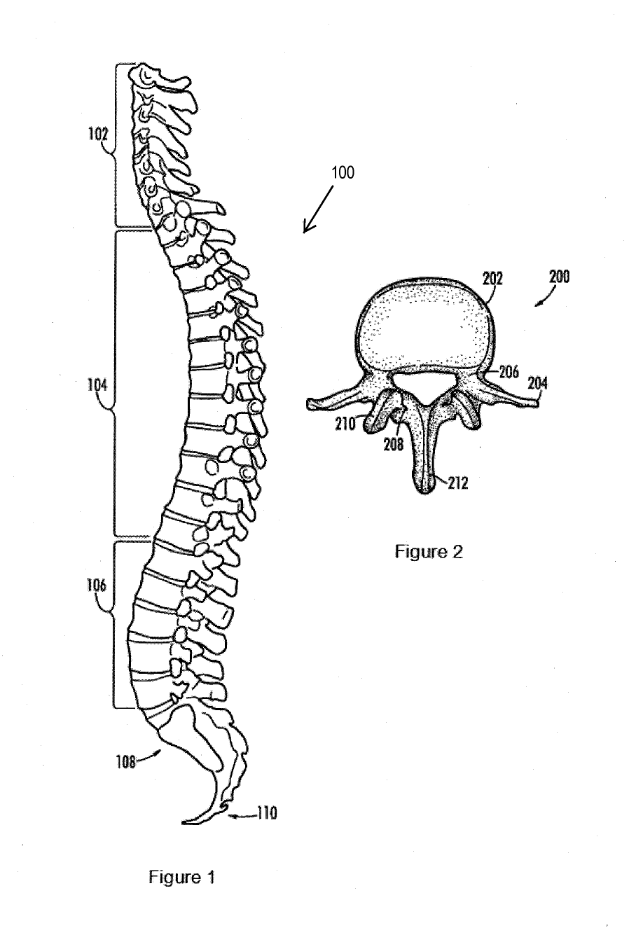

### DETAILED DESCRIPTION

In the following detailed description, reference is made to the accompanying drawings, which form a part of the present disclosure. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and form part of this disclosure. For example, a system or device may be implemented or a method may be practiced using any number of the aspects set forth herein. In addition, such a system or device may be implemented or such a method may be practiced using other structure, functionality, or structure and functionality in addition to or other than one or more of the aspects set forth herein. Alterations and further modifications of the inventive features illustrated herein, and additional applications of the principles of the inventions as illustrated herein, which would occur to one skilled in the relevant art and having possession of this disclosure, are to be considered within the scope of the invention.

Descriptions of unnecessary parts or elements may be omitted for clarity and conciseness, and like reference numerals refer to like elements throughout. In the drawings, the size and thickness of layers and regions may be exaggerated for clarity and convenience.

Features of the present disclosure will become more fully apparent from the following description and appended claims, taken in conjunction with the accompanying drawings. It will be understood these drawings depict only certain embodiments in accordance with the disclosure and, therefore, are not to be considered limiting of its scope; the disclosure will be described with additional specificity and detail through use of the accompanying drawings. An apparatus, system or method according to some of the described embodiments can have several aspects, no single one of which necessarily is solely responsible for the desirable attributes of the apparatus, system or method. After considering this discussion, and particularly after reading the section entitled “Detailed Description” one will understand how illustrated features serve to explain certain principles of the present disclosure.

Embodiments described herein generally relate to systems, devices, and methods for spinal surgeries. More specifically, some embodiments relate to systems, devices, and methods for spinal stabilization.

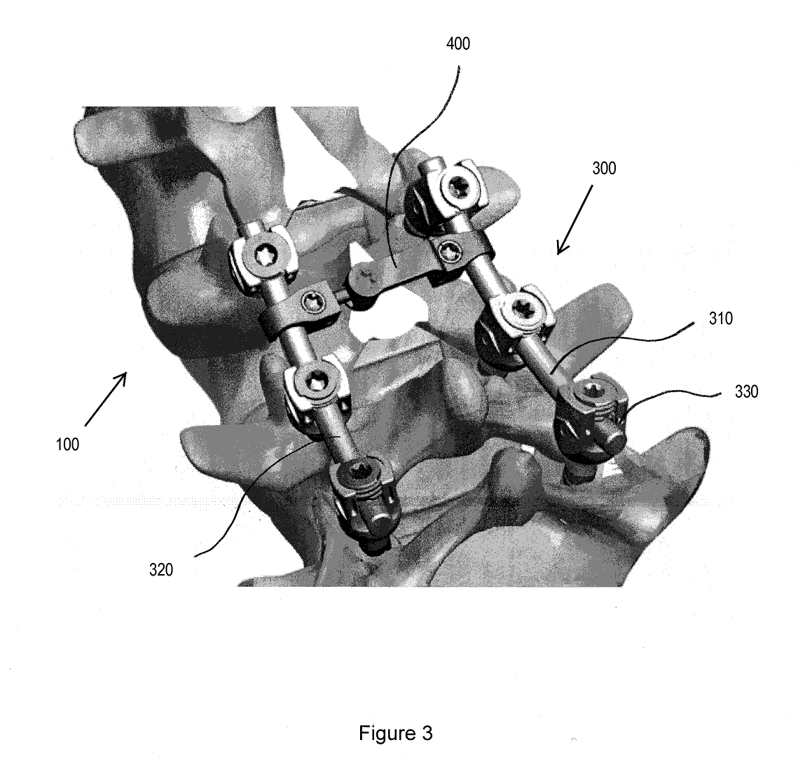

FIG. 3 illustrates a perspective view of one embodiment of a connector 400 coupled to lateral stabilization rods 310, 320 of a spinal stabilization system 300. A spinal stabilization system 300 can include a plurality of pedicle screws 330 and a plurality of spinal stabilization rods 310, 320. In some embodiments, the connector 400 can be configured to couple to a pair of spinal stabilization rods 310, 320. In some embodiments, the connector 400 can be configured to limit movement between the first stabilization rod 310 and the second stabilization rod 320. In some embodiments, the connector 400 can be configured to lock the first stabilization rod 310 in a particular orientation relative to the second stabilization rod 320. In some embodiments, the connector 400 can be configured to rigidly affix the first stabilization rod 310 to the second stabilization rod 320.

In some embodiments, prior to installation of the connector 400, one or more of the vertebral features is removed to effectuate treatment of a patient, for example in a laminectomy at least a portion of a lamina 208 is removed. In some embodiments, some or all of the spinous process 212, the laminae 208, the superior articular processes 210, and/or the transverse processes 204 can be removed in a surgical procedure.

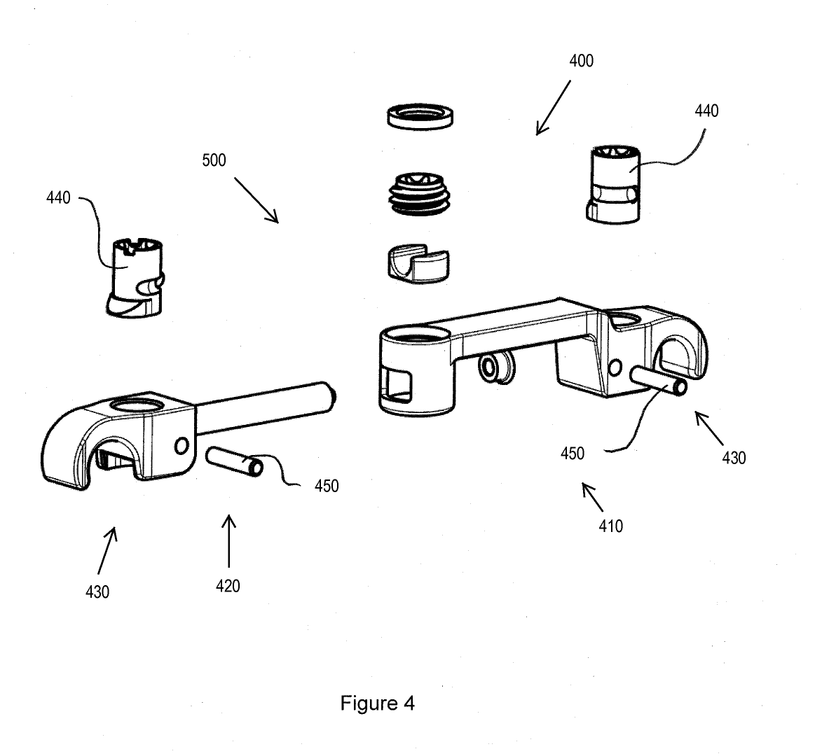

FIG. 4 illustrates an exploded perspective view of one embodiment of a connector 400. In some embodiments, the connector 400 can include a first member 410 and a second member 420. In some embodiments, the first member 410 and second member 420 can each include a coupling portion 430 adapted to couple to a stabilization rod 310, 320. In some embodiments, the first member 410 is adapted to receive the second member 420. In some embodiments, the first member 410 is adapted to lock the second member 420 in position relative to the first member 410. In some embodiments, the second member 420 is slidably positionable within the first member 410. In some embodiments, the second member 420 is rotatably positionable within the first member 410. In some embodiments, the connector 400 can include cam locks 440 configured to block the stabilization rods 310, 320 from uncoupling from the first member 410 and second member 420. In some embodiments, the connector 400 can include guide pins 450 adapted to retain the cam locks 440 within the first member 410 and second member 420.

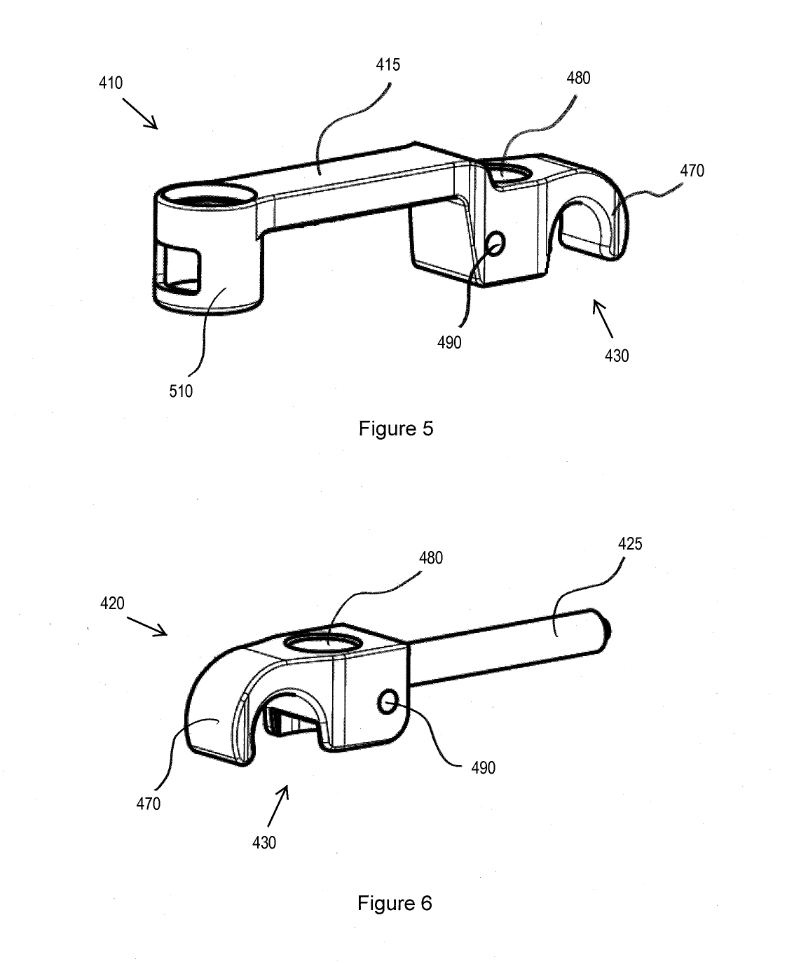

FIG. 5 illustrates a perspective view of one embodiment of a first member 410. In some embodiments, the first member 410 can include a coupling portion 430 adapted to couple to a stabilization rod 310, 320. In some embodiments, the coupling portion 430 of the first member 410 can comprise a hook portion 470 adapted to couple to a first stabilization rod 310. In some embodiments, the first member 410 can include a slide lock housing 510 configured to accept the second member 520. In some embodiments, the coupling portion 430 is connected to the slide lock housing 510 via a connecting portion 415.

FIG. 6 illustrates a perspective view of one embodiment of a second member 420. In some embodiments, the second member 420 can include a coupling portion 430 adapted to couple to a stabilization rod 310, 320. In some embodiments, the coupling portion 430 of the second member 420 can comprise a hook portion 470 adapted to couple to a second stabilization rod 320. In some embodiments, the second member 420 can include a shaft 425. In some embodiments, the shaft 425 is affixed to the coupling portion 430 of the second member 420. In some embodiments, the shaft 425 can be formed integrally with the coupling portion 430 of the second member 420. In some embodiments, the shaft 425 can be inserted into a shaft bore formed in the coupling portion 430 of the second member 420. The shaft 425 can be affixed to the coupling portion 430 in a variety of ways, which can include for example, a press fit, adhesives, welding, etc.

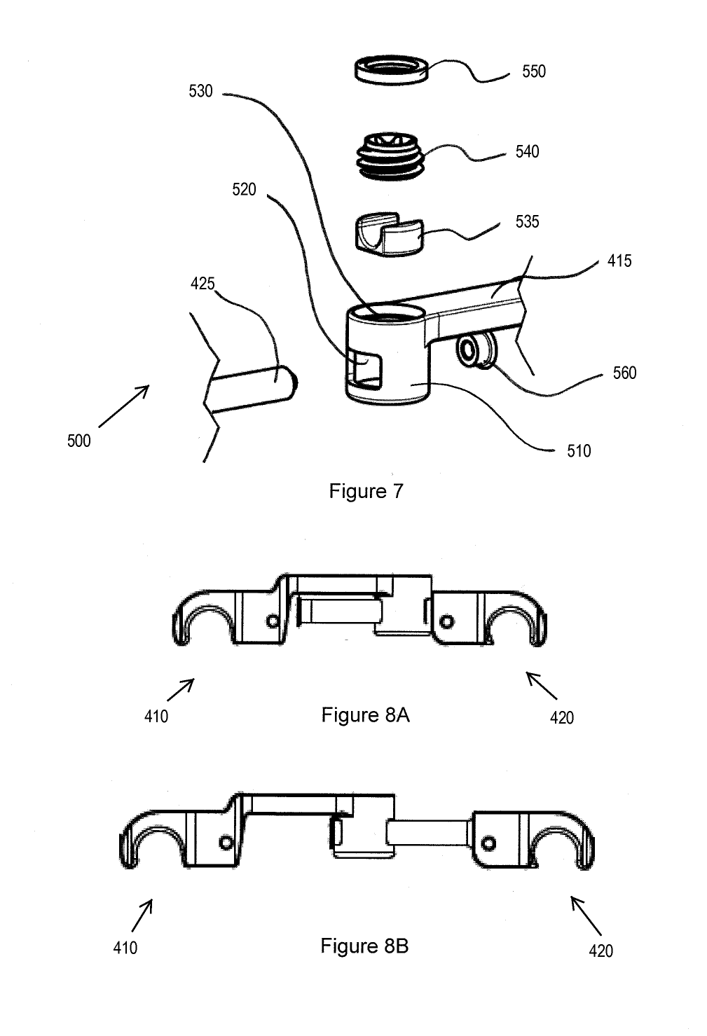

FIG. 7 illustrates an exploded perspective view of one embodiment of a slide lock 500. In some embodiments, the first member 410 can include a slide lock 500 adapted to receive the second member 420 such that the second member 420 is slideably and rotatably positionable within the slide lock 500. In some embodiments, the slide lock 500 can include a slide lock housing 510. In some embodiments, the slide lock housing 510 can be affixed to the first member 410. In some embodiments, the slide lock housing 510 can be integral to the first member 410. The slide lock housing 510 can include a slide window 520 configured to accept the shaft 425 of the second member 420. In some embodiments, the slide lock housing 510 can include a second slide window 520 opposite the slide window 520 configured to accept the shaft 425 of the second member 420. In some embodiments, the slide window 520 and second slide window 520 are wider than the shaft 425, allowing the shaft 425 to rotate within the slide lock housing 510. In some embodiments, the second member 420 can rotate relative to the first member 410 about the axis of the shaft 425. In some embodiments, the second member 420 has three degrees of freedom relative to the first member 410. In some embodiments, the shaft 425 can include a shoulder member 560 on the end of the shaft 425 opposite the coupling portion 430 configured to block the shaft 425 from exiting the slide lock housing 510. In some embodiments, the shoulder member 560 can be affixed to the shaft 425 once the shaft 425 has been inserted through the slide lock housing 510. In some embodiments, the shoulder member 560 can be affixed to the shaft 425 in a variety of ways, which can include for example, a press fit, adhesives, welding, etc.

In some embodiments, the slide lock housing 510 can include a slide bore 530. In some embodiments, the slide bore 530 is substantially perpendicular to the connecting portion 415 of the first member 410. In some embodiments, the slide bore 530 is substantially perpendicular to a stabilization rod 310, 320. In some embodiments, the slide bore 530 is substantially perpendicular to an axis passing through the center of the slide window 520 and second slide window 520. In some embodiments, the slide lock 500 can include a saddle 535 configured to be positioned in the slide bore 530. In some embodiments, the saddle 535 can be adapted to rotate within the slide bore 530. In some embodiments, the saddle 535 can be adapted to slidably receive the shaft 425 of the second member 420. In some embodiments, the saddle 535 can be adapted to center the shaft 425 within the slide bore 530. In some embodiments, the saddle 535 can be adapted to limit rotation of the shaft 425 about a center axis of the slide bore 530. In some embodiments, the slide lock 500 can include a set screw 540. In some embodiments, the slide bore 530 can include an internal thread. In some embodiments, the set screw 540 can be tightened down against the shaft 425 and lock the shaft 425 in place relative to the first member 410. In some embodiments, the slide lock 500 can be adapted to lock the sliding translation of the shaft 425 relative to the first member 410. In some embodiments, the slide lock 500 can be adapted to lock the rotation of the second member 420 relative to the first member 410. In some embodiments, the slide lock 500 can be adapted to lock the sliding translation and the rotation of the second member 420 relative to the first member 410.

In some embodiments, the slide lock 500 can include a cap 550 adapted to retain the set screw 540 within the slide lock housing 510 whether or not the set screw 540 is tightened down. In some embodiments, the cap 550 can be affixed to the slide lock housing 510 after the saddle 535 and set screw 540 are installed in the slide lock housing 510. The cap 550 can be affixed to the slide lock housing 510 in a variety of ways, which can include for example, a press fit, adhesives, welding, etc.

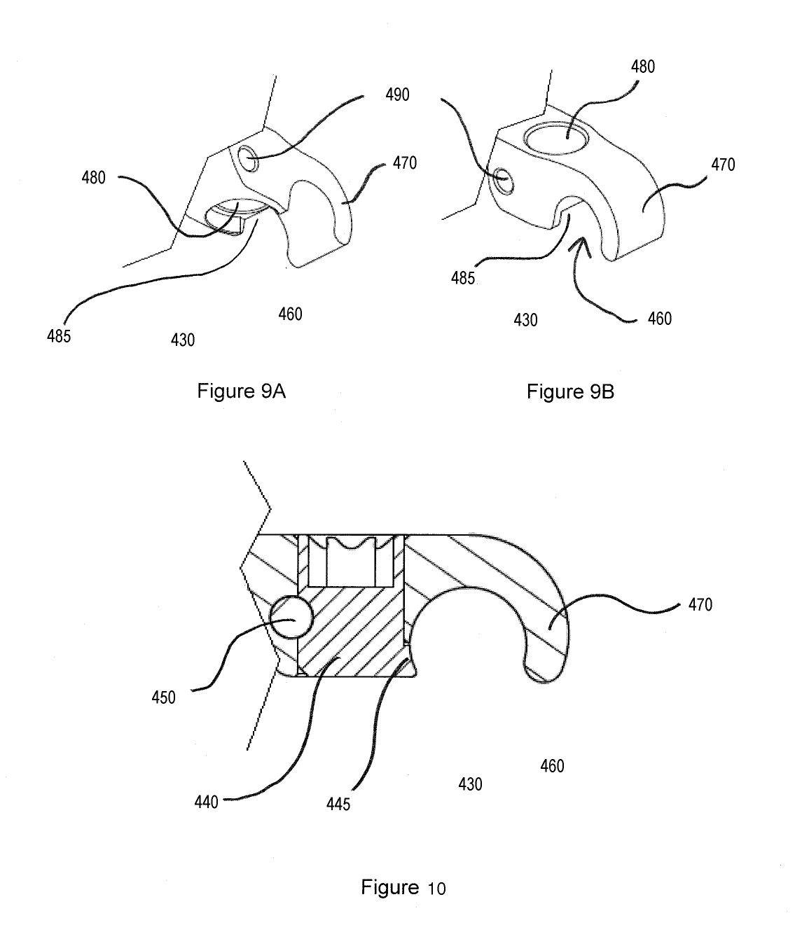

FIG. 8A illustrates a side view of a connector 400 in a short configuration. FIG. 8B illustrates a side view of a connector 400 in a long configuration. In some embodiments, the first member 410 is adapted to slideably receive the second member 420 between a short configuration as illustrated in FIG. 8A and a long configuration as illustrated in FIG. 8B. In some embodiments, the first member 410 can include different length connecting members and the second member 420 can include different length shafts 425 in order to change the distance between the coupling portion 430 of the first member 410 and the coupling portion 430 of the second member 420 in the short and long configurations.

FIG. 9A-B illustrate partial perspective views of one embodiment of a coupling portion 430. In some embodiments, the coupling portion 430 can include a rod recess 460 adapted to receive a stabilization rod 310, 320. In some embodiments, the rod recess 460 can comprise a diameter substantially similar to the diameter of a stabilization rod 310, 320. In some embodiments, the coupling portion 430 can include a hook portion 470 adapted to surround at least a portion of the rod recess 460. In some embodiments, the hook portion 470 is configured to neutrally have a default orientation. In some embodiments, the hook portion 470 is adapted to deflect from a default orientation when the stabilization rod 310, 320 enters the rod recess 460. In some embodiments, the hook portion 470 is adapted to return towards the default orientation when the stabilization rod 310, 320 is fully seated in the rod recess 460. In some embodiments, the hook portion 470 is adapted to retain the stabilization rod 310, 320 in the rod recess 460. In some embodiments, when the stabilization rod 310, 320 is fully seated in the rod recess 460, the coupling portion 430 is adapted to be positioned around greater than 50% of the circumference of the stabilization rod 310, 320. In some embodiments, the coupling portion 430 is adapted to be positioned around approximately 50% to 70% of the circumference of the stabilization rod 310, 320. In some embodiments, when the stabilization rod 310, 320 is fully seated in the rod recess 460, the coupling portion 430 is adapted to be positioned around between 50% and 60% of the circumference of the stabilization rod 310, 320. In some embodiments, when the stabilization rod 310, 320 is fully seated in the rod recess 460, the coupling portion 430 is adapted to be positioned around between 50% and 55% of the circumference of the stabilization rod 310, 320. In some embodiments, when the stabilization rod 310, 320 is fully seated in the rod recess 460, the coupling portion 430 is adapted to be positioned around between 50% and 53% of the circumference of the stabilization rod 310, 320. In some embodiments, when the stabilization rod 310, 320 is fully seated in the rod recess 460, the coupling portion 430 is adapted to be positioned around between 50% and 52% of the circumference of the stabilization rod 310, 320. In some embodiments, the diameter of the rod recess 460 can be greater than the width of the entrance to the rod recess 460. In some embodiments, the diameter of the stabilization rod 310, 320 can be greater than the width of the entrance to the rod recess 460. In some embodiments, the user is able to uncouple the stabilization rod 310, 320 from the connector 400 by forcing the coupling portion 430 away from the stabilization rod 310, 320, such that the hook portion 470 deflects and releases the stabilization rod 310, 320 from the rod recess 460.

In some embodiments, the coupling portion 430 is adapted to couple to a stabilization rod 310, 320 with a snap-fit. In some embodiments, during coupling of the coupling portion 430 to the stabilization rod 310, 320, the deflection and return of the hook portion 470 of the coupling portion 430 can create a snap when the stabilization rod 310, 320 is inserted into the rod recess 460. In some embodiments, the user can force the stabilization rod 310, 320 into the rod recess 460 until the hook portion 470 deflects, allowing the stabilization rod 310, 320 to enter the rod recess 460. In some embodiments, the deflection of the hook portion 470 can produce an abrupt movement of the coupling portion 430 relative to the stabilization rod 310, 320 when the user is forcing the stabilization rod 310, 320 into the rod recess 460. In some embodiments, coupling the coupling portion 430 to the stabilization rod 310, 320 can produce a click. In some embodiments, coupling the coupling portion 430 to the stabilization rod 310, 320 can produce a tactile feel for the user, alerting the user that the connector 400 is coupled to the stabilization rod 310, 320. In some embodiments, coupling the coupling portion 430 to the stabilization rod 310, 320 can produce an audible sound for the user, alerting the user that the connector 400 is coupled to the stabilization rod 310, 320.

In some embodiments, the coupling portion 430 can include a cam bore 480 configured to receive a cam lock 440. In some embodiments, the cam lock 440 can be adapted to be rotatably positioned in the cam bore 480 of the coupling portion 430. In some embodiments, the cam lock 440 is rotatable between a locked position and an open position. In some embodiments, the coupling portion 430 can include a pin bore 490 configured to receive a guide pin 450 (See FIG. 4). In some embodiments, the pin bore 490 can be adapted to receive the guide pin 450 with a press fit. In some embodiments, the pin bore 490 and guide pin 450 can be adapted and positioned to limit axial movement of the cam lock 440 within the cam bore 480. In some embodiments, the guide pin 450 can be adapted to limit rotation of the cam lock 440.

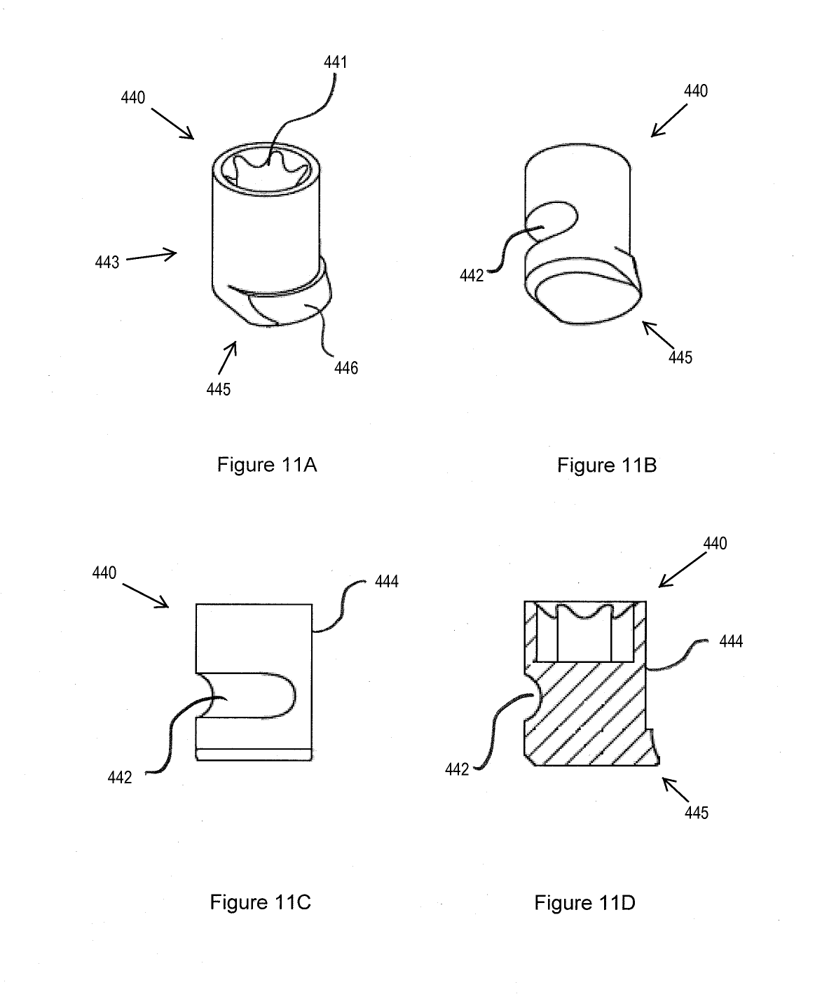

FIG. 10 illustrates a cross-sectional view of one embodiment of a coupling portion 430 and a cam lock 440 in a locked position. FIG. 11A-B illustrate perspective views of one embodiment of a cam lock 440. FIG. 11C illustrates a side view of the cam lock 440 of FIGS. 11A-B. FIG. 11D illustrates a cross-sectional view of the cam lock 440 of FIGS. 11A-B. In some embodiments, the cam lock 440 can include a tool recess 441 adapted to receive a tool and transfer torque from the tool to the cam lock 440. In some embodiments, the cam lock 440 can include a guide channel 442 adapted to slideably receive the guide pin 450. In some embodiments, the guide channel 442 can cooperate with the guide pin 450 to limit axial movement of the cam lock 440 within the cam bore 480. In some embodiments, the guide channel 442 can cooperate with the guide pin 450 to limit rotation of the cam lock 440. In some embodiments, the guide pin 450 can interfere with the end of the guide channel 442 to limit rotation of the cam lock 440. In some embodiments, the coupling portion 430 is adapted to receive a stabilization rod 310, 320 when the cam lock 440 is in an open position. In some embodiments, the coupling portion 430 is adapted to block the stabilization rod 310, 320 from exiting the rod recess 460 when the cam lock 440 is in a locked position, as illustrated in FIG. 10.

In some embodiments, the cam lock 440 can include a cylindrical portion 443. In some embodiments, the cam lock 440 can include a center axis arranged longitudinally along the center of the cylindrical portion 443. In some embodiments, the cylindrical portion 443 can include an outer surface 444. In some embodiments, the outer surface 444 of the cylindrical portion 443 is configured to contact the inner surface of the cam bore 480. In some embodiments, the cam lock 440 can include a protrusion 445 extending outward from the center of the cam lock 440. In some embodiments, the protrusion 445 can extend outwards substantially perpendicular to the center axis of the cam lock 440. In some embodiments, the protrusion 445 can extend outwards past the outer surface 444 of the cylindrical portion 443 of the cam lock 440. In some embodiments, the protrusion 445 only extends outward from a portion of the cam lock 440. In some embodiments, the protrusion 445 can be dimensioned and positioned such that the cam lock 440 engages the stabilization rod 310, 320 when the cam lock 440 is in a locked position but does not engage the stabilization rod 310, 320 when the cam lock 440 is in an open position.

In some embodiments, the coupling portion 430 can include an engagement window 485 configured such that the protrusion 445 of the coupling member can extend through the engagement window 485 when the cam lock 440 is in a locked position (See FIG. 9A-B). In some embodiments, the protrusion 445 of the cam lock 440 is configured to extend through the engagement window 485 when the cam lock 440 is in a locked position and not to extend through the engagement window 485 when the cam lock 440 is in an open position. In some embodiments, the coupling portion 430 is adapted to receive a stabilization rod 310, 320 when the cam lock 440 is in an open position. In some embodiments, the cam lock 440 is adapted to block the stabilization rod 310, 320 from exiting the rod recess 460 when the cam lock 440 is in a locked position, as illustrated in FIG. 10. In some embodiments, the engagement window 485 can be configured to cooperate with the cam lock 440 to limit rotation of the cam lock 440. In some embodiments, the engagement window 485 can be configured to cooperate with the protrusion 445 of the cam lock 440 to limit rotation of the cam lock 440. In some embodiments, the engagement window 485 can be configured such that an edge of the engagement window 485 interferes with the protrusion 445 of the cam lock 440 to limit rotation of the cam lock 440.

In some embodiments, the protrusion 445 of the cam lock 440 can be configured to block the stabilization rod 310, 320 from exiting the rod recess 460. In some embodiments, the protrusion 445 can include a rod engaging surface 446 configured to engage the stabilization rod 310, 320 when the cam lock 440 is in a locked position. In some embodiments, the rod engaging surface 446 can be flat. In some embodiments, the rod engaging surface 446 can be curved. In some embodiments, the diameter of the curve of the rod engaging surface 446 can be adapted to be substantially similar to the diameter of the stabilization rod 310, 320. In some embodiments, the diameter of the curve of the rod engaging surface 446 can be adapted to be substantially similar to the diameter of the rod recess 460. In some embodiments, the rod engaging surface 446 can be configured to align with the rod recess 460, as illustrated in FIG. 10. In some embodiments, the cam lock 440 can advantageously rotate and engage the stabilization rod 310, 320 without producing substantial movement between the stabilization rod 310, 320 and the coupling portion 430. In some embodiments, the cam lock 440 can advantageously rotate and engage the stabilization rod 310, 320 without causing debris. In some embodiments, the cam lock 440 does not utilize a set screw, avoiding any risk of the set screw loosening and the stabilization rod 310, 320 uncoupling form the coupling portion 430.

In some embodiments, the rod engaging surface 446 can be configured such that the connector 400 is positioned around a greater portion of the circumference of the stabilization rod 310, 320 when the cam lock 440 is in a locked position then when the cam lock 440 is in an open position. In some embodiments, the rod engaging surface 446 can be configured such that the connector 400 is positioned around approximately 50% to 90% of the circumference of the stabilization rod 310, 320 when the cam lock 440 is in a locked position. In some embodiments, the rod engaging surface 446 can be configured such that the connector 400 is positioned around approximately 50% to 80% of the circumference of the stabilization rod 310, 320 when the cam lock 440 is in a locked position. In some embodiments, the rod engaging surface 446 can be configured such that the connector 400 is positioned around approximately 50% to 70% of the circumference of the stabilization rod 310, 320 when the cam lock 440 is in a locked position. In some embodiments, the rod engaging surface 446 can be configured such that the connector 400 is positioned around approximately 50% to 60% of the circumference of the stabilization rod 310, 320 when the cam lock 440 is in a locked position. In some embodiments, the rod engaging surface 446 can be configured such that the connector 400 is positioned around approximately 50% to around approximately 75% of the circumference of the stabilization rod 310, 320 when the cam lock 440 is in a locked position. In some embodiments, the rod engaging surface 446 can be configured such that the connector 400 is positioned around approximately 55% to 65% of the circumference of the stabilization rod 310, 320 when the cam lock 440 is in a locked position. In some embodiments, the rod engaging surface 446 can be configured such that the connector 400 is positioned around approximately 58% to 60% of the circumference of the stabilization rod 310, 320 when the cam lock 440 is in a locked position. In some embodiments, when the stabilization rod 310, 320 is fully seated in the rod recess 460, the coupling portion 430 is adapted to be positioned around approximately 51% of the circumference of the stabilization rod 310, 320 when the cam lock 440 is in an unlocked position. In some embodiments, the rod engaging surface 446 can be configured such that the connector 400 is positioned around approximately 59% of the circumference of the stabilization rod 310, 320 when the cam lock 440 is in a locked position.

In some embodiments, the cam lock 440 and coupling portion 430 are configured such that rotation of the cam lock 440 from an open position to a locked position only requires approximately 90 degrees of rotation of the cam lock 440. In some embodiments, the cam lock 440 and coupling portion 430 are configured such that rotation of the cam lock 440 from an open position to a locked position requires approximately 80 to 100 degrees of rotation of the cam lock 440. In some embodiments, the cam lock 440 and coupling portion 430 are configured such that rotation of the cam lock 440 from an open position to a locked position requires approximately 70 to 110 degrees of rotation of the cam lock 440. In some embodiments, the cam lock 440 and coupling portion 430 are configured such that rotation of the cam lock 440 from an open position to a locked position requires approximately 60 to 120 degrees of rotation of the cam lock 440.

In some embodiments, in an open position, the protrusion 445 of the cam lock 440 extends substantially parallel to an axis defined by the rod recess 460. In some embodiments, in a locked position, the protrusion 445 of the cam lock 440 extends substantially perpendicularly to an axis defined by the rod recess 460. In some embodiments, the protrusion 445 is dimensioned such that when the cam lock 440 is in an open position, the protrusion 445 does not interfere with the stabilization rod 310, 320 from entering or exiting the rod recess 460.

In some embodiments, the cam lock 440 can include marking indicia to indicate when the cam lock 440 is in a locked or open position. In some embodiments, the coupling portion 430 can include marking indicia to indicate when the cam lock 440 is in a locked or open position. In some embodiments, the cam lock 440 and coupling portion 430 can be configured such that the cam lock 440 is locked into the locked position once the cam lock 440 is rotated into the locked position. In some embodiments, the cam lock 440 can be configured such that an interference fit exists between the cam lock 440 and the stabilization rod 310, 320 when the cam lock 440 is in a locked position. In some embodiments, the interference fit can limit the cam lock 440 from rotating away from the locked position. In some embodiments, the cam lock 440 can be configured to rotate slightly past the rotational position at which the protrusion 445 projects furthest towards the stabilization rod 310, 320. In some embodiments, the connector 400 can be configured to limit rotation of the cam lock 440 in a first direction to a rotational position slightly past the rotational position at which the protrusion 445 projects furthest towards the stabilization rod 310, 320. In some embodiments, the connector 400 can be configured such that an interference fit exists between the cam lock 440 and the stabilization rod 310, 320 when the cam lock 440 is in a rotational position at which the protrusion 445 projects furthest towards the stabilization rod 310, 320. In some embodiments, the cam lock 440 can be locked into the locked position once the cam lock 440 is rotated in a first direction past the rotational position at which the protrusion 445 projects furthest towards the stabilization rod 310, 320. In some embodiments, the coupling portion 430 and the cam lock 440 can be configured such that the stabilization rod 310, 320 prevents the cam lock 440 from rotating back in a second direction once the cam lock 440 has rotated in a first direction past the rotational position at which the protrusion 445 projects furthest towards the stabilization rod 310, 320. In some embodiments, rotating the cam lock 440 in a first direction past the rotational position at which the protrusion 445 projects furthest towards the stabilization rod 310, 320 can require additional torque compared to simply rotating the cam lock 440 within the cam bore 480. In order to rotate the cam lock 440 in a second direction out of the locked position, additional torque is required to rotate past the rotational position at which the protrusion 445 projects furthest towards the stabilization rod 310, 320. In other embodiments, the guide pin 450 can be arranged such that the guide pin 450 and cam lock 440 bind in a locked position, limiting the cam lock 440 from rotating out of the locked position.

In some embodiments, the first member 410, second member 420, cam locks 440, and/or additional portion of the connector 400 can be made from biocompatible materials which may include, for example, metal, titanium, stainless steel, Nitinol, pyrolitic carbon, polymers, polyether ether ketone, silicone methylmethacrylate, or other biocompatible materials known in the art.

In some embodiments, the first and second members 410, 420 of the connector can be coupled to a respective stabilization rod 310, 320. Coupling the first and second members 410, 420 of the connector to a stabilization rod 310, 320 can include forcing each spinal stabilization rod 310, 320 into each respective rod recess 460 until a portion of the coupling member 430 deflects and each stabilization rod enters a rod recess. In some embodiments, coupling the first and second members 410, 420 of the connector to a stabilization rod 310, 320 can provide a snap-fit. In some embodiments, coupling the first and second members 410, 420 of the connector to a first stabilization rod can provide a tactile feel for the user that the coupling portions 430 have engaged the stabilization rods 310, 320 providing rigid fixation of the connector to each stabilization rod 310, 320. The set screw 540 can be tightened down and locked into place to limit movement between the first member 410 and the second member 420, providing rigid fixation between the stabilization rods 310, 320.

Various modifications to the implementations described in this disclosure may be readily apparent to those skilled in the art, and the principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein. Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of the device as implemented.

Certain features that are described in this specification in the context of separate implementations also can be implemented in combination in a single implementation. Conversely, various features that are described in the context of a single implementation also can be implemented in multiple implementations separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes. However, other operations that are not depicted can be incorporated in the example processes that are schematically illustrated. For example, one or more additional operations can be performed before, after, simultaneously, or between any of the illustrated operations. In certain circumstances, multitasking and parallel processing may be advantageous. Moreover, the separation of various system components in the implementations described above should not be understood as requiring such separation in all implementations. Additionally, other implementations are within the scope of the following claims. In some cases, the actions recited in the claims can be performed in a different order and still achieve desirable results.

In describing the present technology, the following terminology may have been used: The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item. The term “about” means quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3 and 4 and sub-ranges such as 1-3, 2-4 and 3-5, etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

It should be noted that various changes and modifications to the presently preferred embodiments described herein will be apparent to those skilled in the art. Such changes and modifications may be made without departing from the spirit and scope of the invention and without diminishing its attendant advantages. For instance, various components may be repositioned as desired. It is therefore intended that such changes and modifications be included within the scope of the invention. Moreover, not all of the features, aspects and advantages are necessarily required to practice the present invention. Accordingly, the scope of the present invention is intended to be defined only by the claims that follow.

What is claimed is:

1. An adjustable spinal implant device, comprising:
a first member and a second member, the first member adapted to receive the second member and to lock the second member in position relative to the first member;
wherein the first member comprises a first coupling portion adapted to couple to a first spinal stabilization rod with a snap-fit;
wherein the second member comprises a second coupling portion adapted to couple to a second spinal stabilization rod with a snap-fit;
wherein the first coupling portion comprises a first rod recess adapted to receive the first spinal stabilization rod;
wherein the second coupling portion comprises a second rod recess adapted to receive the second spinal stabilization rod;
a first cam lock adapted to be rotatably positioned in the first member,
a second cam lock, adapted to be rotatably positioned in the second member;
wherein the first member comprises a first cam bore configured to receive a first cam lock;
wherein the second member comprises a second cam bore configured to receive a second cam lock;
wherein the first cam lock and second cam lock are each rotatable between a locked position and an open position;
wherein the first rod recess of the first coupling portion is adapted to receive the first spinal stabilization rod when the first cam lock is in an open position;
wherein the first member is adapted to block the first spinal stabilization rod from exiting the rod recess of the first coupling portion when the first cam lock is in a locked position;
wherein the second rod recess of the second coupling portion is adapted to receive the second spinal stabilization rod when the second cam lock is in an open position; and
wherein the second member is adapted to block the second spinal stabilization rod from exiting the rod recess of the second coupling portion when the second cam lock is in a locked position.

2. The spinal implant device of claim 1, wherein the first member comprises a slide lock adapted to receive the second member, wherein the second member is slidably and rotatably positionable within the slide lock.

3. The spinal implant device of claim 1, wherein the first coupling portion comprises a first hook portion, the first hook portion adapted to surround at least a portion of the first rod recess, and wherein the second coupling portion comprises a second hook portion, the second hook portion adapted to surround at least a portion of the second rod recess.

4. The spinal implant device of claim 3, wherein the first hook portion is adapted to deflect from a default orientation when the first spinal stabilization rod enters the first rod recess and to return towards the default orientation when the first stabilization rod is fully seated in the first rod recess, and wherein the second hook portion is adapted to deflect from a default orientation when the second spinal stabilization rod enters the second rod recess and to return towards the default orientation when the second stabilization rod is fully seated in the second rod recess.

5. The spinal implant device of claim 3, wherein the first hook portion is adapted to retain the first spinal stabilization rod in the first rod recess when the first cam lock is in an open position and where the second hook portion is adapted to retain the second spinal stabilization rod in the second rod recess when the second cam lock is in an open position.

6. The spinal implant device of claim 1, wherein the first cam lock comprises a first protrusion adapted to engage the first spinal stabilization rod when the first cam lock is in a locked position, the first protrusion adapted to block the first spinal stabilization rod from exiting the first rod recess, and wherein the second cam lock comprises a second protrusion adapted to engage the second spinal stabilization rod when the second cam lock is in a locked position, the second protrusion adapted to block the second spinal stabilization rod from exiting the second rod recess.

7. The spinal implant device of claim 6, wherein the first protrusion and the second protrusion each comprise a rod engagement surface.

8. The spinal implant device of claim 1, wherein rotation of the first cam lock and the second cam lock from an open position to a locked position requires no more than approximately 100 degrees of rotation.

9. An adjustable spinal implant device, comprising:
a first member and a second member, the first member comprising a slide lock adapted to receive the second member, wherein the second member is slidably and rotatably positionable within the slide lock;
wherein the first member comprises a first coupling portion adapted to couple to a first spinal stabilization rod;
wherein the second member comprises a second coupling portion adapted to couple to a second spinal stabilization rod;
wherein the first coupling portion comprises a first rod recess adapted to receive the first spinal stabilization rod;
wherein the second coupling portion comprises a second rod recess adapted to receive the second spinal stabilization rod;
a first cam lock adapted to be rotatably positioned in the first member,
a second cam lock, adapted to be rotatably positioned in the second member;
wherein the first member comprises a first cam bore configured to receive a first cam lock;
wherein the second member comprises a second cam bore configured to receive a second cam lock;
wherein the first cam lock and second cam lock are each rotatable between a locked position and an open position;
wherein the first rod recess of the first coupling portion is adapted to receive the first spinal stabilization rod when the first cam lock is in an open position;
wherein the first member is adapted to block the first spinal stabilization rod from exiting the rod recess of the first coupling portion when the first cam lock is in a locked position;
wherein the second rod recess of the second coupling portion is adapted to receive the second spinal stabilization rod when the second cam lock is in an open position; and
wherein the second member is adapted to block the second spinal stabilization rod from exiting the rod recess of the second coupling portion when the second cam lock is in a locked position;
wherein rotation of the first cam lock and the second cam lock from a open position to a locked position requires no more than approximately 100 degrees of rotation.

10. The spinal implant device of claim 9, wherein the first cam lock comprises a first protrusion adapted to engage the first spinal stabilization rod when the first cam lock is in a locked position, the first protrusion adapted to block the first spinal stabilization rod from exiting the first rod recess, and wherein the second cam lock comprises a second protrusion adapted to engage the second spinal stabilization rod when the second cam lock is in a locked position, the second protrusion adapted to block the second spinal stabilization rod from exiting the second rod recess.

11. The spinal implant device of claim 10, wherein the first protrusion and the second protrusion each comprise a curved rod engagement surface

12. The spinal implant device of claim 11, wherein the first member comprises a first coupling portion adapted to couple to a first spinal stabilization rod with a snap-fit, and wherein the second member comprises a second coupling portion adapted to couple to a second spinal stabilization rod with a snap-fit.

13. The spinal implant device of claim 12, wherein the first coupling portion comprises a first hook portion, the first hook portion adapted to surround at least a portion of the first rod recess, and wherein the second coupling portion comprises a second hook portion, the second hook portion adapted to surround at least a portion of the second rod recess.

14. The spinal implant device of claim 13, wherein the first hook portion is adapted to deflect from a default orientation when the first spinal stabilization rod enters the first rod recess and to return towards the default orientation when the first stabilization rod is fully seated in the first rod recess, and wherein the second hook portion is adapted to deflect from a default orientation when the second spinal stabilization rod enters the second rod recess and to return towards the default orientation when the second stabilization rod is fully seated in the second rod recess.

15. The spinal implant device of claim 14, wherein the first hook portion is adapted to retain the first spinal stabilization rod in the first rod recess when the first cam lock is in an open position and where the second hook portion is adapted to retain the second spinal stabilization rod in the second rod recess when the second cam lock is in an open position.

16. A method of stabilizing the spine comprising:
coupling a first member of a connector to a first spinal stabilization rod;
coupling a second member of the connector to a second spinal stabilization rod;
rotating a first cam lock approximately 90 degrees from an open position to a locked position; and
rotating a second cam lock approximately 90 degrees from an open position to a locked position.

17. The method of claim 16, further comprising locking the first member to the second member, limiting movement and rotation between the first member and the second member.

18. The method of claim 16, wherein coupling the first member of the connector to the first spinal stabilization rod comprises forcing the first spinal stabilization rod into a first rod recess of the first member until a portion of the first member deflects and the first spinal stabilization rod enters a first rod recess of the first member, and wherein coupling the second member of the connector to the second spinal stabilization rod comprises forcing the second spinal stabilization rod into a second rod recess of the second member until a portion of the second member deflects and the second spinal stabilization rod enters the second rod recess of the second member.

19. The method of claim 16, wherein coupling the first member to the first spinal stabilization rod produces a snap-fit, and wherein coupling the second member to the second spinal stabilization rod produces a snap-fit.

20. The method of claim 16, wherein coupling the first member to the first spinal stabilization rod produces an abrupt movement of the first member relative to the first spinal stabilization rod, and wherein coupling the second member to the second spinal stabilization rod produces an abrupt movement of the second member relative to the second spinal stabilization rod.

21. The method of claim 16, wherein coupling the first member to the first spinal stabilization rod produces a click, and wherein coupling the second member to the second spinal stabilization rod produces a click.
